(19) United States
(12) Patent Application Publication
(10) Pub. No.: US 2017/0202755 A1
(43) Pub. Date: Jul. 20, 2017

(54) ANHYDROUS AEROSOL COMPOSITION BASED ON PARTICLES ENCAPSULATING A BENEFICIAL AGENT

(71) Applicant: L'OREAL, Paris (FR)

(72) Inventors: Gérard MALLE, Villiers S/Morin (FR); Tiina LUUKAS, Sevran (FR); Isabelle BARA, La Verenne St. Hilaire (FR)

(21) Appl. No.: 15/324,079

(22) PCT Filed: Jul. 1, 2015
(86) PCT No.: PCT/EP2015/065003

§ 371 Date: Jan. 5, 2017

(30) Foreign Application Priority Data
Jul. 9, 2014 (FR) 1456629

(51) Int. Cl. A61K 8/11; A61K 8/31; A61K 8/73; B65D 83/14; A61Q 15/00; A61K 8/04; A61Q 13/00; A61Q 5/02; A61K 8/34; A61K 8/60

(52) U.S. Cl. CPC A61K 8/11; A61K 8/345; A61K 8/31; A61K 8/732; A61K 8/738; A61K 8/60; A61Q 15/00; A61K 8/046; A61Q 13/00; A61Q 5/02; B65D 83/752; A61K 2800/624; A61K 2800/87; A61K 2800/412; A61K 2800/31

(57) ABSTRACT

The present invention relates to an anhydrous composition comprising:
1) at least particles releasing a beneficial agent comprising a core containing at least one beneficial agent and an envelope surrounding the core; said envelope comprising at least one hydrophobically modified polysaccharide and at least one water-soluble carbohydrate and/or water-soluble polyol;
   said particles simultaneously having a poured powder density ranging from 300.0 g/l to 600.0 g/l and an absolute density of greater than 1.0 and
2) at least propellant.

The invention also relates to a cosmetic process for caring for and/or for the hygiene of and/or for conditioning and/or for fragrancing and/or for making up a keratin material, which consists in applying to said keratin material a composition as defined previously.

The invention additionally relates to a cosmetic process for treating body odor and optionally human perspiration, which consists in applying to a keratin material a composition as defined previously comprising at least one deodorant active agent and/or antiperspirant active agent in free form and/or in encapsulated form.

The present invention relates to an anhydrous composition comprising:

1) at least particles comprising a core containing at least one beneficial agent and an envelope surrounding the core; said envelope comprising at least one hydrophobically modified polysaccharide and at least one water-soluble carbohydrate and/or water-soluble polyol;
said particles simultaneously having a poured powder density ranging from 300.0 g/l to 600.0 g/l and an absolute density of greater than 1.0 and
2) at least one propellant.

The invention also relates to a cosmetic process for caring for and/or for the hygiene of and/or for conditioning and/or for fragrancing and/or for making up a keratin material, which consists in applying to said keratin material a composition as defined previously.

The invention additionally relates to a cosmetic process for treating body odor and optionally human perspiration, which consists in applying to a keratin material a composition as defined previously comprising at least one deodorant active agent and/or antiperspirant active agent in free form (not encapsulated) and/or in encapsulated form.

Many presentation forms allow the dispensing of beneficial agents, especially of cosmetic or pharmaceutical products, fragrancing products, veterinary products; animal hygiene and/or care products; household maintenance products such as laundry care and/or cleaning products (stain removers, fabric softeners, ironing products), cleaning and/or maintenance products for domestic electrical appliances, cleaning and/or maintenance products for floors, tiles, wood, etc.; sanitary products such as deodorizers, descaling products, unblocking agents for pipes; textile maintenance products, maintenance products for leather goods such as shoes and soles; products derived from the agrifood industry; agricultural products; plant-protection products; paints; inks; maintenance products in the motor vehicle industry.

Among these, aerosols are widely used. Mention will especially be made of household maintenance products, sanitation products, deodorizers, air fresheners and cosmetic products such as deodorants, dry shampoos, hairstyling products or else massage or haircare oils.

It is known that there is a need in many industrial fields to protect a certain number of fragile or volatile molecules, to control their release into an external medium and to be able to produce them in aerosol form.

One of the means for achieving such an aim is to encapsulate them. The object of this encapsulation is to reduce the evaporation and the transfer of the active material toward the environment, either during storage or during the production of the products, or alternatively during their use. Said encapsulation may also make the material easier to use by diluting it and by promoting its uniform distribution in the support.

Microencapsulation especially includes all the technologies for coating or trapping active principles in solid, liquid or gaseous form inside individualized particles whose size ranges between a few microns and a few millimeters. If these microparticles are hollow (vesicular), they are referred to as microcapsules, and if they are filled (matrix-based), they are referred to as microspheres. Their size ranges from 1 μm to more than 1000 μm. These microparticles may or may not be biodegradable and may contain between 5% and 90% (by mass) of active substance.

The encapsulated active substances are of very varied origin: pharmaceutical or cosmetic active principles, food additives, plant protection products, fragranced essences, microorganisms, cells, or alternatively chemical reaction catalysts.

The entire advantage of encapsulation microparticles lies in the presence of a polymer membrane, which isolates and protects the contents from the external medium. Depending on the case, the membrane will be destroyed during use to release its contents (for example: “scratch and sniff” advertising inserts which release perfume when the microcapsules are crushed), or alternatively the membrane will remain present throughout the release of the contents, the rate of diffusion of which it will control (for example: encapsulation of medicaments for sustained release).

The materials constituting the particle are generally hydrophobic or hydrophilic polymers of natural or synthetic origin or alternatively lipids.

The main processes for performing the encapsulation of substances in microparticles are interfacial polymerization, interfacial crosslinking, emulsification followed by evaporation or extraction of the solvent, double emulsification evaporation/extraction of solvent, spray-drying, prilling or coacervation.

The aim of the present invention is to find novel anhydrous aerosol compositions comprising an encapsulated system, for:
encapsulating ingredients that are particularly fragile and volatile while conserving the exact composition developed by the formulator;
providing leaktight particles containing all of the encapsulated ingredients in the absence of stimuli, most particularly for odorous ingredients such as fragrancing ingredients, thus allowing the formulator to combine said capsules with a free perfume of his choice;
to provide particles that are stable in anhydrous media, which can release all or part of their content virtually instantaneously on contact with water and most particularly with atmospheric moisture or perspiration;
to provide particles with a low poured powder density (also known as the loose bulk density), having little tendency to sediment, while at the same time having an absolute density of greater than 1.0, to be able to incorporate them easily into anhydrous vehicles for aerosols;
said particles also needing to be compatible with propellant gases and resistant to compression to be able to be formulated as an aerosol without being damaged. U.S. Pat. No. 5,508,259 proposes nonaqueous fragrancing compositions, especially in aerosol form, comprising perfumes encapsulated in water-soluble spherical particles (also referred to as capsules). Said particles are obtained via conventional encapsulation techniques and in particular the spray-drying of an emulsion formed from a film-forming solid substrate in combination with an emulsifying agent and a mixture of fragrancing ingredients. The film-forming solid substrate is especially chosen from polyvinyl acetate, polyvinyl alcohol, dextrins, natural or modified starch, plant gums, pectins, xanthans, alginates, carrageenans or alternatively cellulose derivatives, for instance carboxymethylcellulose, methylcellulose or hydroxyethylcellulose. The emulsion is then dehydrated via a standard atomization (spray-drying) process, which consists, as described in Example 1 of said patent, in spraying it as fine droplets in an atomizer at a flow rate of 50 kg/h and a pressure of 0.45 bar, in contact with an air stream at 320 m³/h heated to 350° C. so as to evaporate the water, which makes it possible to obtain a fine powder with a particle diameter of between 20 and 80 microns and containing 20% by weight of perfume.

However, it was noted that the particles obtained via this process were highly odorous in dry form on account of the presence of free (non-encapsulated) perfume, that they were formed mainly from agglomerates that were liable to form a sediment in an aerosol device and prevent correct delivery of the product, and that they did not have the density characteristics suitable for the objective of the invention.

U.S. Pat. No. 6,200,949 also describes a process for forming a particulate material containing a hydrophilic perfume, comprising the successive steps consisting in forming an aqueous emulsion of perfume containing 40% to 60% by weight of water, 3% to 30% by weight of maltodextrin and 10% to 40% by weight of hydrophobically modified starch, and then in drying it by spraying in an atomizer (air stream of 420 m³/h heated to 204° C.) so that the particles are formed with a mean size of from about 3 to about 10 microns and a perfume content of from 15% to 50% by weight. However, the particles obtained via this process are highly odorous in dry form on account of the presence of free (non-encapsulated) perfume and are formed mainly from agglomerates which are liable to form a sediment in an aerosol device and prevent correct delivery of the product and do not have the density characteristics suitable for the objective of the invention.

It is thus very important to be able to provide leaktight encapsulation particles which release their contents only on demand (in response to the ambient moisture, especially in humid climatic zones, for example in response to body perspiration, shampooing or showering, etc.), firstly to ensure protection over time of the encapsulated active agent, above all if it is fragile and/or volatile, and 10 secondly to avoid interactions with the other ingredients of the formulation. When the encapsulated beneficial agent is a fragrancing ingredient and/or a whole perfume, it is all the more important for the encapsulation to be total, which leads to odorless particles in anhydrous formulations allowing the formulator to combine them, if desired, with any free perfume of his choice (identical or different) without any risk of interactions or of disruption of the chosen fragranced note.

Patent EP 1 917 098 B1 proposes a process for preparing encapsulation particles by precipitation, said process using:
a pumpable emulsion comprising (i) a continuous phase containing a solvent and a solute forming a matrix dissolved in said solvent and (ii) a dispersed phase;
an extractor comprising a supercritical, subcritical or liquefied gas; said solvent being substantially more soluble in the extractor than said solute forming a matrix, and said process comprising the successive steps consisting in:
   a) combining the pumpable emulsion with the extractor under mixing conditions;
   b) allowing the formation of particulate encapsulation products in which the dispersed phase is embedded in a solid matrix of the solute forming a matrix;
   c) collecting the encapsulation products and separating them from the extractor. It is indicated that this process may be used in the pharmaceutical and agrifood industries and also in the fields of agriculture, coating, adhesives and catalysts. It may be used in particular for encapsulating pharmaceutical active agents, flavorings, enzymes, dyes, pesticides and herbicides.

After extensive research, the Applicant has discovered, surprisingly and unexpectedly, that it is possible to achieve the objectives as stated previously by virtue of an anhydrous aerosol composition containing at least particles comprising a core containing at least one beneficial agent and an envelope surrounding the core; said envelope comprising at least one hydrophobically modified polysaccharide and at least one water-soluble carbohydrate and/or water-soluble polyol; said particles simultaneously having a poured powder density ranging from 300.0 g/l to 600.0 g/l and an absolute density of greater than 1.0. These particles may be obtained in particular via the process as described in patent EP 1 917 098 B1 commented previously.

The particles according to the present invention make it possible, within the anhydrous aerosol compositions, to encapsulate ingredients that are particularly fragile and volatile while conserving the exact composition developed by the formulator. They are leaktight in the absence of stimuli and especially remain odorless for the odorous encapsulated ingredients such as fragrancing ingredients. Said particles are stable in the anhydrous aerosol compositions and can release all or part of their content virtually instantaneously on contact with water and most particularly with atmospheric moisture or perspiration. By virtue of their low poured powder density (loose bulk density), they do not sediment or sediment only very little. They are compatible with propellant gases and are resistant to compression so as to be able to be formulated in an aerosol device without being damaged.

This discovery forms the basis of the present invention.

The present invention relates to an anhydrous composition comprising:

1) at least particles comprising a core containing at least one beneficial agent and an envelope surrounding the core; said envelope comprising at least one hydrophobically modified polysaccharide and at least one water-soluble carbohydrate and/or water-soluble polyol;
said particles simultaneously having a poured powder density ranging from 300.0 g/l to 600.0 g/l and an absolute density of greater than 1.0 and
2) at least one propellant.

Preferably, the composition comprises a physiologically acceptable medium.

According to a particular form of the invention, the compositions of the invention are cosmetic or dermatological.

According to a particular form of the invention, the compositions according to the invention may be used in other industrial applications and may especially be consumer products chosen from veterinary products; animal hygiene and/or care products; household maintenance products such as laundry care and/or cleaning products (stain removers, fabric softeners, ironing products), cleaning and/or maintenance products for domestic electrical appliances, cleaning and/or maintenance products for floors, tiles, wood, etc.; sanitary products such as deodorizers, descaling products, unblocking agents for pipes; textile maintenance products, maintenance products for leather goods such as shoes and soles; products derived from the agrifood industry; agricultural products; plant-protection products; paints; inks; maintenance products in the motor vehicle industry.

The invention also relates to a cosmetic process for caring for and/or for the hygiene of and/or for fragrancing and/or for making up a keratin material, which consists in applying to said keratin material a composition as defined previously.

The invention also relates to a cosmetic process for treating body odor and optionally human perspiration, which consists in applying to a keratin material a composition as defined previously comprising at least one deodorant active agent and/or antiperspirant active agent in free form and/or in encapsulated form.

The invention also relates to an aerosol device formed from a container comprising an aerosol composition as defined previously and from a means for dispensing said composition.

The invention also relates to a consumer product, characterized in that it is conditioned in an aerosol device formed from a container comprising a composition as defined previously and from a means for dispensing said composition.

### Definitions

For the purposes of the present invention, the term “anhydrous composition” means a composition with a water content of less than 5% by weight, preferably less than 2% by weight and even more preferably less than 1% by weight relative to the weight of said composition, or alternatively even less than 0.5% and especially free of water. In this definition, the water mentioned includes the residual water provided by the mixed ingredients.

For the purposes of the present invention, the term “physiologically acceptable medium” is intended to denote a medium that is suitable for the topical administration of a composition. A physiologically acceptable medium is generally a medium which has no unpleasant odor and/or appearance, and which is perfectly compatible with topical administration.

The term “keratin material” means the skin, the scalp, the lips and/or integuments such as the nails and keratin fibers, for instance bodily hair, the eyelashes, the eyebrows and head hair.

For the purposes of the invention, the term “cosmetic composition” means any composition applied to a keratin material to produce a non-therapeutic hygiene, care, conditioning or makeup effect contributing toward improving the well-being and/or enhancing the beauty and/or modifying the appearance of the keratin material onto which said composition is applied.

For the purposes of the invention, the term “dermatological composition” means any composition applied to a keratin material to prevent and/or treat a disorder or dysfunction of said keratin material.

For the purposes of the invention, the term “cosmetic treatment” means any non-therapeutic fragrancing, hygiene, care, conditioning or makeup effect contributing toward improving the well-being and/or enhancing the beauty and/or modifying the appearance or odor of the keratin material onto which said composition is applied.

The term “consumer product” means any manufactured product intended to be used or consumed in the form in which it is sold and which is not intended for a subsequent manufacture or modification. Without the examples being limiting, the consumer products according to the invention may be cosmetic products also including cosmetic formulations for caring for and/or for the hygiene of and/or for making up the skin, the lips, the nails, the eyelashes, the eyebrows, the hair or the scalp; oral hygiene products such as breath fresheners; dermatological products; fragrancing products; pharmaceutical products; products for veterinary use, especially animal hygiene and/or care products; household maintenance products such as laundry care and/or cleaning products (stain removers, fabric softeners, ironing products); products for cleaning and/or maintaining domestic electrical appliances; products for cleaning and/or maintaining floors, tiles, wood, etc.; sanitary products such as deodorizers, descaling products, unblocking agents for pipes; textile maintenance products; maintenance products in leather goods such as shoes and soles; products derived from the agrifood industry; agricultural products; plant-protection products; paints; inks; maintenance products in the motor vehicle industry.

For the purposes of the invention, the term “beneficial agent” means any compound present in a consumer product which produces a beneficial effect perceived by the consumer during its use and/or obtained on the consumer product itself, said beneficial effect possibly being a sensory improvement or a modification, which is especially visual and/or olfactory and/or tactile, an improvement in the comfort and/or ease of application, an esthetic effect, a hygiene effect, a sensation of cleanliness, or a curative and/or prophylactic effect. The term “particles comprising a core containing at least one beneficial agent” means a particle comprising at least one beneficial agent which is immobilized, captured and/or encapsulated in the matrix of an encapsulation or trapping system; said beneficial agent being released to the exterior gradually as the encapsulation or trapping system deteriorates when its degradation takes place on contact with a medium with which it reacts or under the effect of a stimulus such as a supply of water.

### Poured Powder Density (or Loose Bulk Density)

The determination is performed at room temperature (20-25° C.) and under normal atmospheric conditions (1 atmosphere) using a 100 ml measuring cylinder. The measuring cylinder is weighed empty and then filled with a volume of 100 ml of poured powder, without tapping. The difference in mass between the empty measuring cylinder and the cylinder filled with 100 ml of powder gives the poured powder density.

### Absolute Density

### Measurement Principle

The measurement consists in determining the weight of a sample of the solid powder by simple weighing, followed by measuring the volume occupied by the powder particles by measuring the volume of liquid displaced by the powder sample by immersion in this liquid. The liquid chosen must be sparingly volatile and must not be a solvent for the powder. Cyclohexane is generally chosen. The measurements are performed at least twice.

### Materials:

A 10 or 25 ml graduated flask and a precision balance.
m₁ is the weight of the empty flask.
m₂ is the weight of the flask filled with water up to the graduation mark.
m₃ is the weight of the flask filled with cyclohexane up to the graduation mark.
m₄ is the weight of the flask filled to about one third of its volume with the powder to be analyzed.

The flask is filled to about one third of its volume with the powder to be analyzed.

### Method

The flask is filled to slightly below the graduation mark with cyclohexane. In order to completely remove the air trapped in the powder, the following are performed:

1) the flask is treated in an ultrasonic bath for 5 minutes
2) the level of cyclohexane is adjusted to the graduation mark
3) the flask is treated in an ultrasonic bath for 2 minutes
4) steps 2 and 3 are repeated if necessary, until the level of the cyclohexane no longer changes.

m₅ is the weight of the flask thus filled.

The weight of powder analyzed is equal to m₄−m₁ (for good accuracy, this weight must be greater than 2 g). Since the density of air is very low relative to that of the solid, it is taken that m₄−m₁ is equal to the weight of the constituent solid of the powder.

The weight of cyclohexane corresponding to the volume occupied by the solid (Vs) is equal to:

m₆=(m₃−m₁)−(m₅−m₄)=ρ_{cyclo}. Vs where ρ_{cyclo} is the density of cyclohexane at the temperature of the laboratory.

The absolute density of the constituent solid of the powder is equal to ρ_{cyclo}=(m₄−m₁)/Vs=ρ_{cyclo}(m₄−m₁)/m₆.

If the density of cyclohexane at the temperature of the laboratory is unknown, it is determined as follows relative to that of water:

If Vf is the graduated volume of the flask and ρ_{water} is the density of water at the temperature of the laboratory, then:

ρ_{cyclo}=(m₃−m₁)/Vf and ρ_{water}=(m₂−m₁)/Vf

i.e. ρ_{cyclo}=ρ_{water}(m₂−m₁)/(m₃−m₁)

The absolute density of the constituent solid of the powder is equal to:

ρₛ=[ρ_{water}(m₄−m₁)(m₂−m₁)]/[m₆(m₃−m₁)].

### Encapsulation Particles

The particles in accordance with the invention comprise a core containing at least one beneficial agent and an envelope surrounding the core; said envelope comprising at least one hydrophobically modified polysaccharide and at least one water-soluble carbohydrate and/or a water-soluble polyol; said particles simultaneously having a poured powder density ranging from 300.0 g/l to 600.0 g/l and an absolute density of greater than 1.0.

The particles in accordance with the present invention are preferably spherical.

The term “spherical” means that the particle has a sphericity index, i.e. the ratio between its largest diameter and its smallest diameter, of less than 1.2. In this case, such particles are generally referred to as “capsules”.

The term “mean size” of the particles means the parameters D[4,3] and D[2,3] measured via the dry route by laser scattering using a Microtrac S3500 particle size analyzer, the results being expressed in the form of the volume and number particle size distributions giving access to the mean diameter.

The spherical particles in accordance with the present invention thus preferably have a number-mean diameter ranging from 1 to 30 μm, more preferentially ranging from 2 to 15 μm and even better still from 5 to 10 μm and a volume-mean diameter ranging from 5 to 150 μm, preferably ranging from 10 to 100 μm and even better still from 20 to 80 μm.

The particles according to the invention containing the beneficial agent preferably represent from 0.1% to 60% by weight, preferably from 0.3% to 40% by weight and better still from 0.5% to 20% by weight relative to the total weight of the composition.

### Hydrophobically Modified Polysaccharide

The term “hydrophobically modified polysaccharide” means any chemically or enzymatically modified polysaccharide comprising at least one hydrophobic functional group.

Polysaccharides are carbohydrate macromolecules formed by the linking of a large number of hydrophilic elementary sugars (saccharides) bonded together via O-oside bonds.

The hydrophobic functional groups of the present invention are hydrocarbon-based groups (formed essentially from carbon and hydrogen atoms) comprising at least 4 carbon atoms, preferably at least 6 and better still at least 8 carbon atoms, such as alkyl, alkenyl, aryl (i.e. phenyl) or aralkyl (i.e. benzyl) groups. The maximum number of carbon atoms in the hydrocarbon-based group is preferably 24, more preferentially 20 and even more preferentially 18. The hydrophobic hydrocarbon-based groups may be unsubstituted, for example formed from a simple long alkyl chain, or may be substituted with unreactive groups, for instance aromatic groups such as aryl (i.e. phenyl) or aralkyl (i.e. benzyl) groups or alternatively polar groups, for instance carboxyls or hydroxyls.

To graft the hydrophobic functional group(s) onto the polysaccharides, use is generally made of halogenated derivatives, epoxides, isocyanates, or carboxylic acids or derivatives thereof (esters, acid halides or anhydrides).

Among the hydrophobically modified polysaccharides according to the invention, preference is given to hydrophobically modified neutral polysaccharides such as:
celluloses and derivatives thereof, in particular hydrophobically modified methyl-, hydroxyethyl-, ethylhydroxyethyl-, hydroxypropyl-, hydroxypropylmethyl- and carboxymethyl-celluloses. The preferred hydrophobic groups are chosen from C₈-C₁₈ alkyl radicals and more particularly C₁₂-C₁₈ alkyl radicals. In particular, the hydrophobically modified neutral polysaccharides denote hydrophobically modified ethylhydroxyethylcellulose or hydroxyethylcellulose and especially those sold by Ashland under the trade name Natrosol Plus;
hydrophobically modified starches and derivatives thereof (in particular: hydroxyethyl-, hydroxypropyl- and carboxymethyl-starch) and also hydrophobically modified degraded and/or esterified starches,
hydrophobically modified dextrans especially such as the phenoxy-dextrans obtained by reaction between 1,2-epoxy-3-phenoxypropane and a dextran; (C₆-C₁₂)alkyl-dextrans obtained by reaction between 1,2-epoxy-(C₆-C₁₂)alkanes such as 1,2-epoxyoctane or 1,2-epoxydodecane and a dextran;
hydrophobically modified guars and hydroxyethyl-, carboxymethyl- and hydroxypropyl-guar derivatives thereof;
hydrophobically modified pullulans such as cholesterylpullulans;
inulins hydrophobically modified via alkyl ether, ester and carbamate functions, in particular carbamates bearing C₄-C₁₈ alkyl chains and more particularly those sold under the name Inutech® SP1.

The hydrophobically modified polysaccharide preferably represents from 20% to 90% by weight, especially from 30% to 80% by weight, better still from 40% to 70% by weight and even better still from 40% to 60% by weight relative to the total weight of the envelope of the particle.

According to a particularly preferred form of the invention, hydrophobically modified starches will be chosen from among the hydrophobically modified polysaccharides.

The botanical origin of the starch molecules may be cereals or tubers. Thus, the starches are chosen, for example, from corn starch, rice starch, cassava starch, tapioca starch, barley starch, potato starch, wheat starch, sorghum starch and pea starch.

The term “hydrophobically modified starch” means any chemically or enzymatically modified starch comprising at least one hydrophobic functional group.

The hydrophobically modified starches in accordance with the invention are preferably chosen from
C₁₀-C₁₈ hydroxyethyl starch esters and
starch C₅-C₂₀-alkyl or C₅-C₂₀ alkenyl succinates, more particularly C₅-C₂₀-alkenyl succinates and even better still sodium starch octenyl succinate (E1450-CAS 66829-29-6/52906-93-1/70714-61-3), in particular the product sold by National Starch under the name Capsul®.

Mention may also be made of the commercial references Capsul TA®, N-LOK®, N-LOK 1930®, HI-CAP 100®, Purity Gum 1773® and Purity Gum 2000® from National Starch, Cleargum CO® from the company Roquette and Emcap®, Emtex® and Delitex from the company Cargill.

### Water-Soluble Carbohydrate or Polyol

The term “water-soluble carbohydrate” or “water-soluble polyol” refers to a carbohydrate or a polyol which, when introduced into water without pH modification at 25° C., at a mass concentration equal to 3%, makes it possible to obtain a macroscopically homogeneous and transparent solution, i.e. a solution with a minimum light transmittance value, at a wavelength equal to 500 nm, through a sample 1 cm thick, of at least 80% and preferably of at least 90%.

The term “carbohydrates” (also known as saccharides) means all simple sugars or oses and combinations thereof or osides.

Carbohydrates usually comprise:

(1) monosaccharides or oses which are of two types: aldoses comprising an aldehyde function on the first carbon and ketoses comprising a ketone function on the second carbon. They are also distinguished according to the number of carbon atoms they contain.
(2) oligosaccharides (or oligosides), which are saccharide oligomers bearing a sequence of 2 to 10 monosaccharide units linked via glycoside bonds.
(3) polyholosides (or polysaccharides or polyosides), which are saccharide polymers bearing a sequence of more than 10 monosaccharide units.

### Water-Soluble Carbohydrates

### (1) Saccharides or Monosaccharides

Among the saccharides or monosaccharides that may be used according to the invention, mention may be made, alone or as mixtures, of:
tetroses containing four carbons: erythrose, threose, erythrulose;
pentoses containing five carbons: ribose, arabinose, xylose, deoxyribose;
hexoses containing six carbons: glucose, mannose, fucose, gulose, idose, galactose, talose, fuculose, fructose, sorbose, rhamnose;
heptoses containing seven carbons: sedoheptulose in the D and/or L form thereof.

Among the monosaccharides, use will be made more preferentially of arabinose, xylose, fructose, glucose, mannose, rhamnose or threose and even more preferentially glucose or threose.

### (2) Oliqosaccharides

Among the oligosaccharides that may be used according to the invention, mention may be made of:

(i) disaccharides or diholosides or diosides composed of two saccharide molecules.

Among the disaccharides, mention may be made of: cellobiose, isomaltose, isomaltulose, lactose, lactulose, maltose, sucrose, trehalose or melibiose.

(ii) triholosides composed of three saccharide molecules, for instance: raffinose or maltotriose.
(iii) dextrins, which are mixtures of linear glucose oligosides in which the glucose units are linked via oside bonds of the α-(1,4) or α-(1,6) type.
(iv) glucose syrups obtained by acidic or enzymatic hydrolysis of starch, the D.E. of which is between 20 and 100.

D.E. or “dextrose equivalent” is the indicator of the degree of hydrolysis of starch. The higher the D.E., the more extensive the hydrolysis, and thus the higher the proportion of simple (short-chain) sugars.

(v) glucose-fructose syrups especially with a high content of fructose (HFCS: high-fructose corn syrup), which denote a series of corn syrups that have been subjected to enzymatic processes in order to increase their fructose content before being mixed with glucose syrup to obtain their final composition.

Among the glucose-fructose syrups, also known as isoglucose syrups, which may be used according to the invention, mention may be made of:
HFCS 90, which contains 90% fructose and 10% glucose syrup;
HFCS 55, which contains 55% fructose and 45% glucose syrup;
HFCS 42, which contains 42% fructose and 58% glucose syrup.

Among the oligosaccharides, use will be made more preferentially of cellobiose, maltose, isomaltose, raffinose and glucose syrups, more particularly glucose syrups.

Use will be made preferentially of a glucose syrup with a D.E. ranging from 21 to 60 and even more preferentially a glucose syrup with a D.E. of from 21 to 38, for instance the dehydrated glucose syrups sold by Tereos under the names G210, G290 and G380.

### (3) Polysaccharides or Polyholosides

Examples that may be mentioned include:
dextrans, which are composed of D-glucose units linked via an α(1-6) oside bond and bearing branches formed from alpha-1,2 or 1,3 or 1,4 bonds. They are prepared by fermentation of beet sugar solely containing hydroxyl groups. It is possible to obtain dextran fractions of different molecular weights from native dextran by hydrolysis and purification. The dextran may in particular be in the form of dextran sulfate.
pullulans, which are formed from maltotriose units, known under the name α(1,4)-α(1,6)-glucan. Three glucose units in maltotriose are connected via an α-(1,4) glycoside bond, whereas the consecutive maltotriose units are connected to each other via an α-(1,6) glycoside bond. It is produced from starch by the fungus Aureobasidium pullulans. Pullulan is produced, for example, under the reference Pullulan PF 20® by the group Hayashibara in Japan.
maltodextrins, which are the result of hydrolysis of a cereal (i.e.: wheat, corn) starch or of a tuber (i.e.: potato) starch. They are formed from various sugars (i.e.: glucose, maltose, maltotriose, oligosaccharides and polyosides) derived directly from this reaction, in proportions which depend on the degree of hydrolysis.

This degree is measured by the “dextrose equivalent”, or D.E., dextrose or D-glucose being the result of a total hydrolysis of starch. The higher the D.E., the more extensive the hydrolysis, and thus the higher the proportion of simple (short-chain) sugars of which maltodextrin is composed.

The maltodextrins used in accordance with the invention preferentially have a D.E. ranging from 4 to 20 and better still maltodextrins with a D.E. ranging from 12 to 20.

Use will preferably be made of potato or corn maltodextrins such as those sold under the trade names MD 20P® from Avebe and Maldex 120®, Maldex 170® and Maldex 190® from Tereos.

### Polyols

For the purposes of the invention, polyols are linear, branched and/or cyclic, non-glycoside, saturated or unsaturated carbon-based and especially hydrocarbon-based compounds, comprising 4 to 18 carbon atoms, especially 4 to 16, or even 4 to 12 carbon atoms, and 3 to 9 hydroxyl (OH) groups, and also possibly comprising one or more oxygen atoms intercalated in the chain (ether function). The polyols in accordance with the invention are preferably linear or branched saturated hydrocarbon-based compounds, comprising 4 to 18 carbon atoms, especially 4 to 16 or even 4 to 12 carbon atoms, and 3 to 9 hydroxyl (OH) groups.

They may be chosen, alone or as mixtures, from:
triols, such as trimethylolethane or trimethylolpropane;
tetraols such as pentaerythritol (tetramethylolmethane), erythritol, diglycerol or ditrimethylolpropane;
pentols such as arabitol;
hexols such as dulcitol, sorbitol, mannitol, dipentaerythritol or triglycerol;
heptols such as volemitol;
octaols;
nonanols such as isomalt, maltitol, isomaltitol or lactitol.

Preferably, the polyol is chosen from sorbitol, maltitol, mannitol and isomalt, and mixtures thereof.

Among the water-soluble carbohydrates and water-soluble polyols in accordance with the invention, the ones that will more particularly be chosen are water-soluble oligo- and polysaccharides and more preferentially dextrans, pullulans, glucose syrups and maltodextrins and better still glucose syrups with a D.E. ranging from 21 to 38 and/or maltodextrins with a D.E. ranging from 4 to 20 and better still maltodextrins with a D.E. ranging from 12 to 20.

Use will preferably be made of glucose syrups such as those sold by Tereos under the names G210, G290 and G380 and potato or corn maltodextrins such as those sold under the trade names MD 20P® from Avebe and Maldex 120®, Maldex 170® and Maldex 190® from Tereos.

The water-soluble carbohydrate(s) and/or polyol(s) in accordance with the invention represent from 10% to 80% by weight, preferably from 15% to 70% by weight, more preferentially from 20% to 65% by weight and better still from 40% to 60% by weight relative to the total weight of the envelope of the particle.

According to a particularly preferred form of the invention, the envelope of the particles according to the invention is formed from
at least one starch (C₅-C₂₀)alkenyl succinate and
at least one maltodextrin with a D.E. ranging from 4 to 20 and preferably ranging from 12 to 20 and/or a glucose syrup with a D.E. ranging from 21 to 60 and preferentially from 21 to 38.

According to a first variant, the envelope of the particles according to the invention is formed from at least one starch (C₅-C₂₀)alkenyl succinate and from at least one maltodextrin with a D.E. ranging from 4 to 20 and preferably ranging from 12 to 20.

According to a second variant, the envelope of the particles according to the invention is formed from at least one starch (C₅-C₂₀)alkenyl succinate and from at least one glucose syrup with a D.E. ranging from 21 to 60 and preferentially ranging from 21 to 38.

According to a particularly preferred form of the invention, the envelope of the encapsulation particles is formed from

a) at least one starch (C₅-C₂₀)alkenyl succinate in an amount ranging from 20% to 90% by weight, especially from 30% to 80% by weight, preferably from 40% to 70% by weight and better still from 40% to 60% by weight relative to the total weight of the envelope of the particle and
b) at least one glucose syrup with a D.E. ranging from 21 to 38 and/or a maltodextrin with a D.E. ranging from 4 to 20 in an amount ranging from 10% to 80% by weight, preferably from 15% to 70% by weight, more preferentially from 20% to 65% by weight and better still from 40% to 60% by weight relative to the total weight of the envelope of the particle.

According to a particularly preferred form of the invention, the envelope of the encapsulation particles is formed from:

a) at least one starch (C₅-C₂₀)alkenyl succinate in an amount ranging from 20% to 90% by weight, especially from 30% to 80% by weight, preferably from 40% to 70% by weight and better still from 40% to 60% by weight relative to the total weight of the envelope of the particle and
b) at least one maltodextrin with a D.E. ranging from 4 to 20 in an amount ranging from 10% to 80% by weight, preferably from 15% to 70% by weight, more preferentially from 20% to 65% by weight and better still from 40% to 60% by weight relative to the total weight of the envelope of the particle.

According to a particularly preferred form of the invention, the envelope of the encapsulation particles is formed from:

a) at least one starch (C₅-C₂₀)alkenyl succinate in an amount ranging from 20% to 90% by weight, especially from 30% to 80% by weight, preferably from 40% to 70% by weight and better still from 40% to 60% by weight relative to the total weight of the envelope of the particle, and
b) at least one glucose syrup with a D.E. ranging from 21 to 38 in an amount ranging from 10% to 80% by weight, preferably from 15% to 70% by weight, more preferentially from 20% to 65% by weight and better still from 40% to 60% by weight relative to the total weight of the envelope of the particle.

### Process for Preparing the Encapsulation Particles

The particles according to the invention may especially be prepared according to the process described in patent EP 1 917 098 B1 from FeyeCon.

According to a particular form of the invention, the particles are obtained according to a process comprising at least the following steps:
an aqueous solution formed from a mixture of the water-soluble carbohydrate and/or the water-soluble polyol and of the hydrophobically modified polysaccharide is prepared, the beneficial agent is then added and the whole is stirred so as to form an emulsion; and
said emulsion thus formed is homogenized at high pressure at a pressure ranging from 10 to 200 bar and more preferentially from 20 to 200 bar;
said emulsion is sprayed, preferably continuously, in a drying chamber; and
the water is extracted for a time preferably not exceeding 3 hours, and more preferentially not exceeding 30 minutes, with a fluid under pressure such as carbon dioxide, preferably in supercritical form, preferably at a pressure of at least 0.3 XPc and at a temperature of at least Tc−60° C. with Pc corresponding to the critical pressure of the gas and Tc the critical temperature of the gas, so as to obtain particles, which are preferably spherical, with a mean size preferably ranging from 1 to 150 μm, more preferentially ranging from 2 to 100 μm and better still from 5 to 80 μm.

### Propellant

As previously indicated, the composition comprises one or more propellants.

The propellant used in the cosmetic composition according to the invention is chosen from dimethyl ether, volatile hydrocarbons such as propane, isopropane, n-butane, isobutane, n-pentane and isopentane, and mixtures thereof, optionally with at least one chlorinated and/or fluorinated hydrocarbon; among the latter, mention may be made of the compounds sold by the company DuPont de Nemours under the names Freon® and Dymeli®, and in particular monofluorotrichloromethane, difluorodichloromethane, tetrafluorodichloroethane and 1,1-difluoroethane sold especially under the trade name Dymel 152 A® by the company DuPont.

Carbon dioxide, nitrous oxide, nitrogen or compressed air may also be used as propellant gas.

Preferably, the composition according to the invention comprises a propellant chosen from volatile hydrocarbons.

More preferentially, the propellant is chosen from dimethyl ether, propane, isopropane, n-butane, isobutane, pentane and isopentane, and mixtures thereof. The weight ratio between the composition without propellant (fluid) and the propellant preferably varies in a ratio from 5/95 to 60/40, preferably from 10/90 to 50/50 and more preferentially from 15/85 to 30/70.

Another subject of the present invention is an aerosol device formed from a container comprising a composition as defined previously and a means for dispensing said composition.

The dispensing means, which forms a part of the aerosol device, is generally formed from a dispensing valve controlled by a dispensing head, itself comprising a nozzle via which the aerosol composition is vaporized. The container containing the pressurized composition may be opaque or transparent. It may be made of glass, polymer or metal, optionally coated with a layer of protective varnish.

The devices in accordance with the invention are well known to those skilled in the art and comprise aerosol containers comprising a propellant and also aerosol pumps using compressed air as propellant. These devices are described in patents U.S. Pat. No. 4,077,441 and U.S. Pat. No. 4,850,517.

### Beneficial Agents

The amount of beneficial agent present in the particles in accordance with the invention preferably ranges from 0.1% to 80% by weight relative to the weight of the particle, preferably from 1% to 70% by weight, better still from 10% to 60% and even better still from 15% to 50% by weight relative to the total weight of the particle.

The time for release of the beneficial agent will obviously vary according to the nature and intensity of the stimulus.

The total duration for release of the beneficial agent may be modified and will depend greatly on the composition of the aerosol formula, the content of particles present in the aerosol, the nature and especially the chemical nature of the beneficial agent and its concentration in the particles (amount encapsulated in the particle) and the nature and intensity of the stimulus to which the particle containing the beneficial agent will be subjected. The release may equally be instantaneous or last several hours or even several days.

Among the beneficial agents that may be used according to the invention, mention may be made more particularly of:

(i) fatty substances;
(ii) fragrancing substances;
(iii) pharmaceutical active principles;
(iv) cosmetic active agents.

### Fatty Substances

Fatty substances are often used in the formulation of. They may be chosen from the group comprising

(i) natural oils of plant, animal or marine origin,
(ii) mineral oils,
(iii) hydrogenated oils,
(iv) silicone oils,
(v) terpenes,
(vi) squalene,
(vii) saturated or unsaturated fatty acids,
(viii) fatty acid esters,
(x) waxes,
(x) fatty alcohols,
(xi) butters such as shea butter or cocoa butter,
(xii) and mixtures thereof.

### Fragrancing Substances

The term “fragrancing substance” means any ingredient that is capable of giving off a pleasant odor.

Perfumes are compositions especially containing starting materials (generally referred to as perfumery ingredients) described in S. Arctander, Perfume and Flavor Chemicals (Montclair, N.J., 1969), in S. Arctander, Perfume and Flavor Materials of Natural Origin (Elizabeth, N.J., 1960) and in Flavor and Fragrance Materials—1991, Allured Publishing Co., Wheaton, Ill.

They may be synthesis products or natural products, for instance essential oils, absolutes, resinoids, resins, concretes, and/or synthetic products (terpene or sesquiterpene hydrocarbons, alcohols, phenols, aldehydes, ketones, ethers, acids, esters, nitriles or peroxides, which may be saturated or unsaturated, and aliphatic or cyclic).

According to the definition given in international standard ISO 9235 and adopted by the Commission of the European Pharmacopoeia, an essential oil is an odoriferous product generally of complex composition, obtained from a botanically defined plant raw material, either by steam entrainment, or by dry distillation, or via an appropriate mechanical process without heating. The essential oil is generally separated from the aqueous phase via a physical process which does not result in any significant change in the composition.

Among the essential oils that may be used according to the invention, mention may be mode of those obtained from plants belonging to the following botanical families:

Abietaceae or Pinaceae: conifers; Amaryllidaceae; Anacardiaceae; Anonaceae: ylang ylang; Apiaceae (for example Umbelliferae): dill, angelica, coriander, sea fennel, carrot, parsley; Araceae; Aristolochiaceae; Asteraceae: yarrow, artemisia, camomile, helichrysum; Betulaceae; Brassicaceae; Burseraceae: frankincense; Caryophyllaceae; Canellaceae; Cesalpiniaceae: copaifera (copaiba balsam); Chenopodaceae; Cistaceae: rock rose; Cyperaceae; Dipterocarpaceae; Ericaceae: gaultheria (wintergreen); Euphorbiaceae; Fabaceae; Geraniaceae: geranium; Guttiferae; Hamamelidaceae; Hernandiaceae; Hypericaceae: St John's wort; Iridaceae; Juglandaceae; Lamiaceae: thyme, oregano, monarda, savory, basil, marjorams, mints, patchouli, lavenders, sages, catnip, rosemary, hyssop, balm; Lauraceae: ravensara, sweet bay, rosewood, cinnamon, litsea; Liliaceae: garlic; lily, lily of the valley, hyacinth, daffodil; Magnoliaceae: magnolia; Malvaceae; Meliaceae; Monimiaceae; Moraceae: hemp, hop; Myricaceae; Myristicaceae: nutmeg; Myrtaceae: eucalyptus, tea tree, paperbark tree, cajuput, backhousia, clove, myrtle; Oleaceae; Piperaceae: pepper; Pittosporaceae; Poaceae: lemon balm, lemongrass, vetiver; Polygonaceae; Renonculaceae; Rosaceae: roses; Rubiaceae; Rutaceae: all citrus plants; Salicaceae; Santalaceae: sandalwood; Saxifragaceae; Schisandraceae; Styracaceae: benzoin; Thymelaceae: agarwood; Tilliaceae; Valerianaceae: valerian, spikenard; Verbenaceae: lantana, verbena; Violaceae; Zingiberaceae: galangal, turmeric, cardamom, ginger; Zygophyllaceae.

Mention may also be made of the essential oils extracted from flowers (lily, lavender, rose, jasmine, ylang ylang, neroli), from stems and leaves (patchouli, geranium, petitgrain), from fruit (raspberry, peach, coriander, aniseed, cumin, juniper), from fruit peel (bergamot, lemon, orange, grapefruit), from roots (angelica, celery, cardamom, iris, sweet flag, ginger), from wood (pinewood, sandalwood, gaiac wood, rose of cedar, camphor), from grasses and gramineae (tarragon, rosemary, basil, lemongrass, sage, thyme), from needles and branches (spruce, fir, pine, dwarf pine) and from resins and balms (galbanum, elemi, benzoin, myrrh, olibanum, opopanax).

Examples of fragrancing substances are especially: geraniol, geranyl acetate, farnesol, borneol, bornyl acetate, linolool, linalyl acetate, linalyl propionate, linalyl butyrate, tetrahydrolinolool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, cis-3-hexenyl acetate, vetiveryl acetate, ethyl acetate, butyl acetate, hexyl acetate, decyl acetate, isoamyl acetate, stearyl acetate, allyl heptanoate, vetiverol, α-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, allyl 3-cyclohexylpropionate, ethyl 6-(acetyloxy)hexanoate, allyl caproate, ethyl 2-methylbutyrate, methyl dihydrojasmonate, hexyl salicylate, 4-(4-hydroxy-4-m ethylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranyl acetone, n-decanal, n-dodecanal, anisylpropanal, 9-decen-1-ol, cis-3-hexenol, tetrahydro-2-isobutyl-4-methylpyran-4-ol, 3-methyl-5-phenyl-1-pentanol, 3a,6,6,9α-tetramethyldodecahydronaphtho[2,1-b]furan, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotropin, coumarin, eugenol, vanillin, diphenyl ether, citral, citronellal, hydroxycitronellal, hexylcinnamal, 2,4-dimethylcyclohex-3-ene-1-carbaldehyde, 2,6-dimethylhept-5-enal, α,α-dimethyl-p-ethylphenylpropanal, 1,3-benzodioxole-5-carboxaldehyde, limonene, damascone, decalactone, nonalactone, 6,6-dimethoxy-2,5,5-trimethylhex-2-ene, 2,4,4,7-tetramethyloct-6-en-3-one, 1-(5,5-dimethyl-1-cyclohexenyl)pent-4-en-1-one, methylheptenone, 4-(cyclopropylmethyl)phenyl methyl ether, 2-methyl-6-methylideneoct-7-en-2-ol, rose oxide, 1-(1,2,3,4,5,6,7,8-octahydro-2,3,8,8-tetramethyl-2-naphthyl)ethan-1-one, 2-acetonaphthone, 2-isopropyl-5-methylcyclohexanone, ionones, methylionones, isomethylionones, solanone, irones, cis-3-hexenol and esters thereof, indane musks, tetralin musks, isochroman musks, macrocyclic ketones, macrolactone musks, aliphatic musks, ethylene brassylate, rose essence, and mixtures thereof.

In general, perfumes are formed from a mixture of perfumery ingredients which may also be classified into head notes, heart notes and base notes.

The three notes correspond to the greater or lesser volatility of the ingredients of which they are composed: highly volatile head note, moderately volatile heart note and sparingly volatile base note.

(i) The head note, also known as the “top” note, is that which is first perceived by the sense of smell as soon as the perfume comes into contact with the keratin material or any substrate. However, it is the note which fades the fastest: it does not “last”. It is difficult to express the time of persistence of this note, since it is very variable: from a few minutes to about 10 minutes.

It is essentially fresh and light. All the citrus notes especially fall into this category. In perfumery, they are grouped under the generic term hesperidean notes, which include orange, lemon, grapefruit, bergamot, neroli, etc. Mention will also be made of herbal notes such as lavender, laurel, thyme or rosemary, and aniseed, menthol, aldehyde, etc. notes. Mention will also be made of eucalyptus notes.

(ii) The heart note, also occasionally referred to as the “body note”, has a persistence which lasts from a few tens of minutes to a few hours, but its main characteristic is that it is not perceived until after a few minutes. Thus, it “starts” just before the head note dies off. It begins to express itself while the head note is gradually fading away. It is represented essentially by floral, fruity or spicy scents: lily of the valley, honeysuckle, violet, magnolia, cinnamon, geranium, jasmine, rose, iris, raspberry, peach, etc.
(ii) The base note, also occasionally known as the “bottom note”, gives a perfume its “durability”, persistence or staying power. It is perceptible several hours, or even several days, or even several weeks after application onto clothing or a perfume blotter or scent strip, depending on the concentration of the perfume. Examples that will be mentioned include woods, roots, mosses and resins and animal or mineral substances such as opoponax, musks, amber, sandalwood, benzoin, lichen, clove, sage, etc. Mention will also be made of vanilla, patchouli, coumarin, etc. notes.

Needless to say, ingredients belonging to one or more notes may be encapsulated. However, it will be preferred to encapsulate the most volatile ingredients (i.e. the least persistent) belonging to the head and/or heart notes. Among these ingredients, examples that will be mentioned include:

benzyl acetate
geranyl acetate
cis-3-hexenyl acetate
C18 aldehyde or nonalactone
decyl acetate
allyl amyl glycolate (citral)
ethyl acetate
butyl acetate
allyl 3-cyclohexylpropionate
linalyl acetate
phenylethyl alcohol
hexyl acetate
Berryflor or ethyl 6-(acetyloxy)hexanoate
isoamyl acetate
allyl caproate
Amarocite or 6,6-dimethoxy-2,5,5-trimethylhex-2-ene
Citral Lemarome N or 3,7-dimethylocta-2,6-dienal
Canthoxal or anisylpropanal
Claritone or 2,4,4,7-tetramethyloct-6-en-3-one
ethyl 2-methylbutyrate
dihydromyrcenol
cis-3-hexenol
Hedione or methyl dihydrojasmonate

### L-carvone

allyl heptanoate
limonene
Neobutenone Alpha or 1-(5,5-dimethyl-1-cyclohexenyl)pent-4-en-1-one

### Methylheptenone

Toscanol or 4-(cyclopropylmethyl)phenyl methyl ether
Myrcenol Super or 2-methyl-6-methylideneoct-7-en-2-ol
decalactone
stearyl acetate
rose oxide
linalool
Triplal or 2,4-dimethylcyclohex-3-ene-1-carbaldehyde
Melonal or 2,6-dimethylhept-5-enal
1-(1,2,3,4,5,6,7,8-octahydro-2,3,8,8-tetramethyl-2-naphthyl)ethan-1-one hexylcinnamal
tetrahydro-2-isobutyl-4-methylpyran-4-ol
hexyl salicylate
1,4-dioxacycloheptadecane-5,17-dione
and mixtures thereof.

According to a particular form of the invention, the encapsulation particles comprise at least one or more fragrancing substances with a saturating vapor pressure at 25° C. of greater than or equal to 10.0 Pa will preferably be chosen.

The saturating vapor pressure (or vapor tension) is the pressure at which the gaseous phase of a substance is in equilibrium with its liquid or solid phase at a given temperature in a closed system. Calculation of the saturating vapor pressure may be performed using the following formula:

$$ln \frac{{p}_{sat}}{{p}_{0}} = \frac{M . {L}_{v}}{R} ( \frac{1}{{T}_{0}} - \frac{1}{T} )$$

with:
T₀: boiling point of the substance at a given pressure p₀, in degrees Kelvin,
pₛₐₜ: saturating vapor pressure, in the same unit as p₀
M: molar mass of the substance, in kg/mol
Lᵥ: latent heat of vaporization of the substance, in joules/kg
R: ideal gas constant, equal to 8.31447 J/K/mol
T: temperature of the vapor, in K.

Preferably, the fragrancing substances with a saturating vapor pressure at 25° C. of greater than or equal to 10 Pa represent an amount ranging from 50% to 100% by weight, preferably from 60% to 100% by weight, more preferentially from 70% to 100% by weight and better still from 80% to 100% by weight relative to the total weight of the fragrancing substances present in the particles of the invention.

### Pharmaceutical Active Principles

The term “pharmaceutical active principle” means a molecule or a mixture of molecules which has a curative and/or prophylactic therapeutic effect, which can be administered by spraying.

### Cosmetic Active Agents

The term “cosmetic active agent” means any molecule which has a hygiene, care, makeup or coloring effect contributing toward the improvement well-being and/or enhancement or modification of the appearance of the human keratin material onto which said composition is applied.

Among the cosmetic active agents that may be applied to human keratin materials such as the skin, the lips, the scalp, the hair, the eyelashes or the nails, examples that may be mentioned, alone or as mixtures, include:
vitamins and derivatives or precursors thereof, alone or as mixtures,
antioxidants;
cleaning agents such as surfactants;
dyestuffs;
conditioning agents;
agents for relaxing and/or straightening and/or shaping the hair;
free-radical scavengers;
photoprotective agents such as organic or mineral UV-screening agents;
self-tanning agents;
anti-glycation agents;
calmatives;
hair-removing agents;
deodorant agents;
antiperspirant agents;
NO-synthase inhibitors;
agents for stimulating fibroblast proliferation;
agents for stimulating keratinocyte proliferation;
dermo-relaxing agents;
refreshing agents;
tensioning agents;
matt-effect agents;
skin-shine counteractants;
antiseborrhea agents;
greasy-hair counteractants;
depigmenting agents;
pro-pigmenting agents;
keratolytic agents;
desquamating agents;
moisturizers;
antimicrobial agents;
slimming agents;
agents that act on the energy metabolism of cells;
insect repellents;
substance P or CGRP antagonists;
hair-loss counteractants;
antiwrinkle agents;
antiaging agents;
antidandruff agents

Among these cosmetic active agents, preference will be given most particularly, alone or as mixtures, to:
photoprotective agents such as UV-screening agents, in particular organic UV-screening agents;
skin-shine counteractants;
antiseborrhea agents;
greasy-hair counteractants;
deodorant agents;
antiperspirant agents;
refreshing agents;
matt-effect agents;
antimicrobial agents;
antidandruff agents

According to a particularly preferred form of the invention, the beneficial agent(s) present in the particles will be chosen from fragrancing substances.

According to an even more particularly preferred form of the invention, the fragrancing substances present in the particles are chosen from heart notes and/or head notes so as to be able both to compensate for their loss throughout the day and to afford an additional freshness effect throughout the day in response to perspiration or to atmospheric humidity or humidity provided, for example, by misters.

According to a particular form of the invention, the composition will contain particles containing at least one fragrancing substance and at least one fragrancing substance in free form, which may be identical to or different from the fragrancing substance present in said particles.

Said fragrancing substances in free form may be chosen from those mentioned previously.

According to another particular form of the invention, the composition exclusively contains the fragrancing substance(s) in the encapsulation particles. In other words, all of the ingredients for fragrancing that are present in the composition are contained in the particles.

The composition according to the invention may advantageously comprise at least one fatty phase which may comprise at least one compound chosen from volatile or non-volatile, carbon-based, hydrocarbon-based, fluoro and/or silicone oils, waxes and/or solvents of mineral, animal, plant or synthetic origin, alone or as a mixture, provided that they form a stable homogeneous mixture and are compatible with the intended use.

For the purposes of the invention, the term “volatile” means any compound that is capable of evaporating on contact with the keratin substances in less than one hour, at room temperature (25° C.) and atmospheric pressure (1 atm). This volatile compound has especially a nonzero vapor pressure, at room temperature and atmospheric pressure, especially ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Conversely, the term “nonvolatile” means a compound that remains on the human keratin substances at room temperature and atmospheric pressure for at least one hour, and that especially has a vapor pressure of less than 10⁻³ mmHg (0.13 Pa).

The composition may also comprise other ingredients in free form (not encapsulated or imprisoned in the particles of the invention) used commonly in cosmetic compositions. Such ingredients may be chosen from antioxidants, preserving agents, cosmetic active agents such as those mentioned previously, fragrancing substances such as those described previously, surfactants, spreading agents, wetting agents, dispersants, antifoams, neutralizers, stabilizers, polymers and especially liposoluble film-forming polymers, and mixtures thereof.

Needless to say, those skilled in the art will take care to select this or these optional additional compound(s) and/or the amounts thereof so that the advantageous properties of the composition for the use according to the invention are not, or are not substantially, adversely affected by the envisaged addition.

The compositions according to the invention may be in any form that is acceptable and common for a composition intended to be conditioned in an aerosol device.

A person skilled in the art can choose the appropriate composition, and also its method of preparation, on the basis of his general knowledge, taking into account first the nature of the constituents used, especially their solubility in the support, and secondly the application envisaged for the composition.

According to a particular form of the invention, the compositions according to the invention are fragrancing products conditioned in an aerosol device such as an eau de toilette, a perfume, an ambience perfume in which the particles comprise at least one fragrancing substance. More particularly, the compositions will also contain a fragrancing substance in free form, which may be identical to or different from the fragrancing substance present in the particles.

According to another particular form of the invention, the compositions according to the invention may be in the form of a hair product conditioned in an aerosol device. These hair products may especially be products intended for non-rinsed cleansing of the hair such as dry shampoos, intended for hairstyling and/or hairsetting such as hair lacquers, mousses or conditioning products; disentangling, sheen, smoothing, etc. cosmetic products for caring for and/or treating the hair and the scalp. More particularly, the particles comprise at least one fragrancing substance.

Even more particularly, the compositions will also contain at least one fragrancing substance in free form, which may be identical to or different from the fragrancing substance(s) present in the particles.

According to another particular form of the invention, the compositions according to the invention may be in the form of a hygiene product, in particular of deodorants and/or antiperspirants in which the composition comprises at least one deodorant active agent and/or at least one antiperspirant active agent, in free form and/or in encapsulated form. More particularly, the particles comprise at least one fragrancing substance. Even more particularly, the compositions will also contain at least one fragrancing substance in free form, which may be identical to or different from the fragrancing substance(s) present in the particles.

### Antiperspirant Active Agent

The term “antiperspirant active agent” means a compound which, by itself, has the effect of reducing the flow of sweat and/or of reducing the sensation of moisture on the skin associated with human sweat and/or of partially or totally absorbing human sweat.

Among the antiperspirant active agents that may be mentioned are aluminum and/or zirconium salts such as aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, alum salts, aluminum sulfate, aluminum zirconium octachlorohydrate, aluminum zirconium pentachlorohydrate, aluminum zirconium tetrachlorohydrate, aluminum zirconium trichlorohydrate and more particularly the aluminum chlorohydrate in activated or nonactivated form sold by the company Reheis under the name Microdry Aluminum Chlorohydrate® or by the company Guilini Chemie under the name Aloxicoll PF 40. Aluminum and zirconium salts are, for example, the product sold by the company Reheis under the name Reach AZP-908-SUF®, “activated” aluminum salts, for example the product sold by the company Reheis under the name Reach 103 or by the company Westwood under the name Westchlor 200.

Preferably, the cosmetic composition comprises aluminum chlorohydrate as antiperspirant active agent.

As other antiperspirant active agent, mention may be made of expanded perlite particles such as those obtained by the expansion process described in patent U.S. Pat. No. 5,002,698.

The perlites that may be used according to the invention are generally aluminosilicates of volcanic origin and have the composition:

70.0%-75.0% by weight of silica SiO₂
12.0%-15.0% by weight of aluminum oxide Al₂O₃
3.0%-5.0% of sodium oxide Na₂O
3.0%-5.0% of potassium oxide K₂O
0.5%-2% of iron oxide Fe₂O₃→
0.2%-0.7% of magnesium oxide MgO
0.5%-1.5% of calcium oxide CaO
0.05%-0.15% of titanium oxide TiO₂.

Preferably, the perlite particles used will be ground; in this case, they are known as Expanded Milled Perlite (EMP). They preferably have a particle size defined by a median diameter D50 ranging from 0.5 to 50 μm and preferably from 0.5 to 40 μm.

Preferably, the perlite particles used have a loose bulk density at 25° C. ranging from 10 to 400 kg/m³ (standard DIN 53468) and preferably from 10 to 300 kg/m³.

Preferably, the expanded perlite particles according to the invention have a water absorption capacity, measured at the wet point, ranging from 200% to 1500% and preferably from 250% to 800%.

The wet point corresponds to the amount of water which has to be added to 100 g of particles in order to obtain a homogeneous paste. This method is directly derived from the oil uptake method applied to solvents. The measurements are taken in the same manner by means of the wet point and the flow point, which have, respectively, the following definitions:

Wet point: weight, expressed in grams per 100 g of product, corresponding to the production of a homogeneous paste during the addition of a solvent to a powder.

Flow point: mass expressed in grams per 100 g of product above which the amount of solvent is greater than the capacity of the powder to retain it. This is reflected by the production of a more or less homogeneous mixture which flows over the glass plate.

The wet point and the flow point are measured according to the following protocol:

### Protocol for Measuring the Water Absorption

### 1) Equipment Used

Glass plate (25×25 mm)
Spatula (wooden shaft and metal part, 15×2.7 mm)
Silk-bristled brush

### Balance

### 2) Procedure

The glass plate is placed on the balance and 1 g of perlite particles is weighed out. The beaker containing the solvent and the sampling pipette is placed on the balance. The solvent is gradually added to the powder, the whole being regularly blended (every 3 to 4 drops) by means of the spatula.

The mass of solvent needed to obtain the wet point is noted. Further solvent is added and the mass which makes it possible to reach the flow point is noted. The average of three tests will be determined.

The expanded perlite particles sold under the trade names Optimat 1430 OR or Optimat 2550 by the company World Minerals will be used in particular.

### Deodorant Active Agents

The term “deodorant active agent” refers to any substance that is capable of masking, absorbing, improving and/or reducing the unpleasant odor resulting from the decomposition of human sweat by bacteria.

The deodorant active agents may be bacteriostatic agents or bactericides that act on underarm odor microorganisms, such as 2,4,4′-trichloro-2′-hydroxydiphenyl ether (®Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (®Triclocarban) or 3,7,11-trimethyldodeca-2,5,10-trienol (®Farnesol); quaternary ammonium salts such as cetyltrimethylammonium salts, cetylpyridinium salts; polyols such as those of glycerol type, 1,3-propanediol (Zemea Propanediol® sold by DuPont Tate & Lyle Bio Products), 1,2-decanediol (Symclariol® from the company Symrise), glycerol derivatives, for instance caprylic/capric glycerides (Capmul MCM® from Abitec), glyceryl caprylate or caprate (Dermosoft GMCY® and Dermosoft GMC®, respectively from Straetmans), polyglyceryl-2 caprate (Dermosoft DGMC® from Straetmans), biguanide derivatives, for instance polyhexamethylene biguanide salts; chlorhexidine and salts thereof; 4-phenyl-4,4-dimethyl-2-butanol (Symdeo MPP® from Symrise); cyclodextrins; chelating agents such as Tetrasodium Glutamate Diacetate (CAS #51981-21-6) sold under the trade name Dissolvine GL-47-S® from AkzoNobel, EDTA (ethylenediaminetetraacetic acid) and DPTA (1,3-diaminopropanetetraacetic acid).

Among the deodorant active agents in accordance with the invention, mention may also be made of:
zinc salts, such as zinc salicylate, zinc phenolsulfonate, zinc pyrrolidonecarboxylate (more commonly known as zinc pidolate), zinc sulfate, zinc chloride, zinc lactate, zinc gluconate, zinc ricinoleate, zinc glycinate, zinc carbonate, zinc citrate, zinc chloride, zinc laurate, zinc oleate, zinc orthophosphate, zinc stearate, zinc tartrate, zinc acetate or mixtures thereof;
odor absorbers such as zeolites, especially silver-free metal zeolites, cyclodextrins, metal oxide silicates such as those described in patent application US 2005/063 928; metal oxide particles modified with a transition metal, as described in patent applications US 2005/084 464 and US 2005/084 474, aluminosilicates such as those described in patent application EP 1 658 863, chitosan-based particles such as those described in patent U.S. Pat. No. 6,916,465;
sodium bicarbonate;
salicylic acid and derivatives thereof such as 5-n-octanoylsalicylic acid;
alum;
triethyl citrate;

The deodorant active agents may preferably be present in the compositions according to the invention in weight proportions of from 0.01% to 10% by weight relative to the total weight of the composition.

The invention also relates to a cosmetic process for treating body odor and optionally human perspiration, which consists in applying to a keratin material a composition comprising particles as defined previously; said composition comprising at least one deodorant active agent and/or at least one antiperspirant active agent in free form and/or in encapsulated form.

The invention is illustrated in greater detail in the examples that follow.

Examples of Preparing Particles with Release of Perfume

### EXAMPLE A

Capsules were prepared using the following composition:

| | Composition | | | |
|---|---|---|---|---|
| | Hydrophobically | Water-soluble | | |
| Example A | modified starch | polysaccharide | Fragrance* | Water |
| | Amidon Capsul ® | Potato maltodextrin | 55 g | 225 g |
| | from National | MD 20 P from | | |
| | Starch | Avebe | | |
| | 110 g | 110 g | | |

*The perfume used has the following composition:

| Ingredients | Amount in g |
|---|---|
| Isopropyl myristate | 20.5 |
| Methyl dihydrojasmonate | 15 |
| 2-Phenylethanol | 8 |
| 1-(1,2,3,4,5,6,7,8-Octahydro- | 8 |
| 2,3,8,8-tetramethyl- | |
| 2-naphthyl)ethan-1-one | |
| Hexylcinnamal | 6 |
| Tetrahydro-2-isobutyl-4- | 6 |
| methylpyran-4-ol | |
| Hexyl salicylate | 6 |
| Benzyl acetate | 5 |
| 1,4-Dioxacycloheptadecane- | 5 |
| 5,17-dione | |
| 3-Methyl-5-phenyl-1-pentanol | 5 |
| Dihydromyrcenol | 4 |
| Orange terpenes 0.05% B H T | 4 |
| (limonene >95%) | |
| 2-Acetonaphthone | 2 |
| 3a,6,6,9a- | 1 |
| Tetramethyldodecahydronaphtho[2,1- | |
| b]furan | |
| α, α-Dimethyl-p-ethylphenylpropanal | 1 |
| 1,3-Benzodioxole-5-carboxaldehyde | 1 |
| 2-Isopropyl-5-methylcyclohexanone | 1 |
| 1-Phenylethyl acetate | 0.8 |
| 2,6-Dimethylhept-5-enal (Melonal) | 0.5 |
| 2,4-Dimethylcyclohex-3-ene-1- | 0.2 |
| carbaldehyde (Triplal) | |

### Process for Preparing the Emulsion

Potato maltodextrin MD20 P and Amidon Capsul® (sodium salt of starch octenyl succinate) were mixed in water until dissolved, the perfume was then added and the whole was emulsified with a Heidolph Diax 900 Ultra-Turrax disperser (motor power 900 W with an electronically controlled speed of 8000 to 26 000 rpm) at the maximum power for 4 minutes.

### Drying Procedure for Obtaining Spherical Particles

The emulsion obtained was then homogenized at a pressure of 30 bar using a high-pressure pump and then sprayed in an atomization chamber using a nozzle simultaneously with a stream of CO₂ (30 bar, 45° C.) which was circulated continuously at a flow rate of about 500 g/min to remove the water. The dried powder was retained on a filter located at the base of the atomization chamber, and then collected after depressurization. 270 g of spherical microcapsules are thus obtained in the form of a fine white powder with a number-mean diameter of 7.8 μm and a volume-mean diameter of 47 μm.

The particle size was measured via a dry route by laser scattering using a Microtrac S3500 particle size analyzer, the particle sizes being expressed by volume and by number.

| | Measured characteristics of the capsules | | | |
|---|---|---|---|---|
| | Amount of | Amount | | |
| | encapsulated | of free | Poured | |
| | perfume | perfume | powder | Absolute |
| Example A | (%) | (%) | density | density |
| | 19.8 | <0.1 | 484 | 1.12 |

### EXAMPLES B TO H

According to the process described in Example A, the following capsules were prepared:

| | Composition | | | | |
|---|---|---|---|---|---|
| | Hydrophobically | | Perfume | | |
| | modified | Water-soluble | of | | |
| | starch | polysaccharide | Example A | | Water |
| Example B | Amidon Capsul ® | Maltodextrin MD | 55 | g | 225 g |
| | from National | 120 from Tereos | | | |
| | Starch | 110 g | | | |
| | 110 g | | | | |
| Example C | Amidon Capsul ® | Maltodextrin MD | 55 | g | 225 g |
| | from National | 170 from Tereos | | | |
| | Starch | 110 g | | | |
| | 110 g | | | | |
| Example D | Amidon Capsul ® | Maltodextrin MD | 55 | g | 225 g |
| | from National | 190 from Tereos | | | |
| | Starch | 110 g | | | |
| | 110 g | | | | |
| Example E | Amidon Capsul ® | Potato maltodextrin | 105 | g | 225 g |
| | from National | MD 20 P from | | | |
| | Starch | Avebe | | | |
| | 110 g | 110 g | | | |
| Example F | Amidon Capsul ® | Potato maltodextrin | 55 | g | 225 g |
| | from National | MD 20 P from | | | |
| | Starch | Avebe 66 g | | | |
| | 154 g | | | | |
| Example G | Amidon Capsul ® | Potato maltodextrin | 55 | g | 225 g |
| | from National | MD 20 P from | | | |
| | Starch 66 g | Avebe | | | |
| | | 154 g | | | |
| Example H | Amidon Capsul ® | Glucose syrup | 55 | g | 225 g |
| | from National | Glucodry G290 | | | |
| | Starch | from Tereos | | | |
| | 110 g | 110 g | | | |

| | Measured characteristics of the capsules | | | |
|---|---|---|---|---|
| | | Amount | Poured | |
| | Amount of | of free | powder | |
| | encapsulated | perfume | density | Absolute |
| Examples | perfume (%) | (%) | (g/l) | density |
| Example | 19.3 | <0.1 | 568 | 1.14 |
| B | | | | |
| Example | 19.4 | <0.1 | 490 | 1.16 |
| C | | | | |
| Example | 19.9 | <0.1 | 537 | 1.11 |
| D | | | | |
| Example | 38 | 0.8 | 482 | 1.08 |
| E | | | | |
| Example | 21.0 | 0.2 | 595 | 1.11 |
| F | | | | |
| Example | 20.7 | 0.2 | 521 | 1.15 |
| G | | | | |
| Example | 19.2 | 0.1 | 568 | 1.12 |
| H | | | | |

### COMPARATIVE EXAMPLE I

Capsules having the same composition as Example A as described above were prepared according to the process of Example 1 of patent U.S. Pat. No. 6,200,949 comprising drying by spray-drying (atomization) of the emulsion described in Example A.

The emulsion is dried by spray-drying using a Bowen Lab Model Dryer machine using air with a flow rate of 420 m³/h at a temperature of 204° C. and an external temperature of 93° C. and a turbine speed of 50 000 rpm.

Morphological aspect of the particles obtained: polymorphous with aggregates

### COMPARATIVE EXAMPLE J

Capsules having the same composition as Example A as described above were prepared according to the process of Example 1 of patent U.S. Pat. No. 5,508,259 comprising drying by spray-drying (atomization) of the emulsion described in Example A.

The mixture was dried by spray-drying with a CCM Sulzer machine at an emulsion flow rate of 50 kg/h, air at a flow rate of 320 m³/h at 350° C. and 0.45 bar.

Morphological Aspect of the Particles Obtained: Polymorphous with Aggregates

| | Measured characteristics of the capsules | | | |
|---|---|---|---|---|
| | | Amount | Poured | |
| | Amount of | of free | powder | |
| | encapsulated | perfume | density | Absolute |
| Composition | perfume (%) | (%) | (g/l) | density |
| Example I | 18.3 | 2.7 | 259 | 1.16 |
| (outside the | | | | |
| invention) | | | | |
| Example J | 11.2 | 1.7 | 269 | 1.12 |
| (outside the | | | | |
| invention) | | | | |

### EXAMPLE 1: DEODORANT AND ANTIPERSPIRANT PRODUCT

An anhydrous antiperspirant aerosol having the following composition was prepared:

| Ingredients | (weight %) |
|---|---|
| Dimethicone | 23.51 |
| Isopropyl palmitate | 4.85 |
| Dimethicone dimethiconol | 8.90 |
| Triethyl citrate | 5.66 |
| Modified bentonite | 2.10 |
| Anhydrous aluminum | 28.30 |
| hydroxychloride | |
| Perfume capsules of | 26.68 |
| Example A | |
| Total | 100.00 |

35.3 g of dimethicone, 7.3 g of isopropyl palmitate, 13.3 g of dimethicone/dimethiconol and 8.5 g of triethyl citrate are placed in a tank equipped with a stirrer and a bottom turbomixer. The mixture is homogenized with vigorous stirring (1000 rpm). 3.1 g of modified bentonite are then added and the mixture is left to stand for 10 minutes. The mixture is homogenized again with vigorous stirring (1200 rpm), followed by the addition of 42.4 g of anhydrous aluminum hydroxychloride. The mixture is homogenized again with vigorous stirring (1200 rpm), followed by the addition of 40.0 g of capsules of Example A. A thick opaque beige-white liquid was thus obtained, which is introduced into a closed aerosol can. The air is removed from the container, which is then filled with the propellant gas (isobutane) in a 15 (fluid)/85 (gas) ratio.

### EXAMPLES C1 AND C2: DEODORANT AND ANTIPERSPIRANT PRODUCTS

In a manner similar to that of Example 1, anhydrous antiperspirant aerosols having the following compositions were prepared:

Example C1

| Ingredients | (weight %) |
|---|---|
| Dimethicone | 23.51 |
| Isopropyl palmitate | 4.85 |
| Dimethicone dimethiconol | 8.90 |
| Triethyl citrate | 5.66 |
| Modified bentonite | 2.10 |
| Anhydrous aluminum | 28.30 |
| hydroxychloride | |
| Perfume capsules of | 26.68 |
| Example I | |
| Total | 100.00 |

Example C2

| Ingredients | (weight %) |
|---|---|
| Dimethicone | 23.51 |
| Isopropyl palmitate | 4.85 |
| Dimethicone dimethiconol | 8.90 |
| Triethyl citrate | 5.66 |
| Modified bentonite | 2.10 |
| Anhydrous aluminum | 28.30 |
| hydroxychloride | |
| Perfume capsules of | 26.68 |
| Example J | |
| Total | 100.00 |

### Evaluation of the Examples:

Comparison of the Formulations of Example 1 with the Formulations of Examples C1 and C2 Before Pressurization Under Gas

| Aerosol fluid | Appearance | Density* |
|---|---|---|
| Example 1 | Thick homogeneous | 1.153 |
| | beige-white liquid | |
| Example C1 | Thick non-homogeneous | Not |
| | beige-white paste | measurable |
| Example C2 | Thick non-homogeneous | Not |
| | beige-white paste | measurable |

*the density is measured using a Mettler-Toledo DA-100M densimeter. A sample of the homogenized fluid is taken using a 5 ml syringe and introduced into the densimeter. The machine automatically indicates the density.

### COMPARISON OF THE AEROSOLS: EXAMPLE 1, EXAMPLE C1 AND EXAMPLE C2

| | Sedimentation | | Appearance | Spraying (0.41 |
|---|---|---|---|---|
| Aerosol | time | | of the fluid | mm nozzle) |
| Example 1 | 25 | s | 0 | OK |
| Example C1 | 15 | s | 2 | Repeated |
| | | | | clogging of |
| | | | | the nozzle |
| | | | | then gas |
| | | | | leaks |
| Example C2 | 13 | s | 3 | OK |

The criteria for evaluating the sedimentation time are the following:

≧25 s=slow sedimentation
15-24 s=moderate sedimentation
≦14 s=rapid sedimentation

The criteria for the appearance of the fluid are the following:

0=no particles visible in the fluid (fluid is completely translucent)
1=small presence of particles visible in the fluid
2=moderate presence of particles visible in the fluid
3=strong presence of particles visible in the fluid
4=very strong presence of particles visible in the fluid
* reference DSPR119 from Precision.

### Evaluation Protocol:

The aerosol is shaken for three seconds to homogenize it. It is then sprayed for 2 seconds onto a scent strip (Granger Veyron reference: 40140 BCSI of size 4 cm×14 cm) so as to deposit about 0.07 g of composition. After 1 minute, the olfactory intensity is evaluated (BEF) and is graded from 0 to 10. Perspiration is then stimulated by adding about 0.1 g of water (three sprays) onto the deposited composition. After waiting for 30 seconds, the olfactory intensity is evaluated (AFT). 4 hours later, it is re-evaluated again before and after spraying the same amount of water. 20 hours later, the intensity before/after spraying with water is re-evaluated again in the same manner.

| | Odor | | | Odor | | | Odor intensity | | |
|---|---|---|---|---|---|---|---|---|---|
| | intensity T0 | | | intensity T4 h | | | T24 h | | |
| Aerosol | BEF | AFT | Δ | BEF | AFT | Δ | BEF | AFT | Δ |
| Example 1 | 0 | 6 | 6 | 2.7 | 6.7 | 4.0 | 1.3 | 4.0 | 2.7 |
| Example C1 | 6 | 8 | 2 | 6.7 | 8.0 | 1.3 | 4.7 | 5.3 | 0.7 |
| Example C2 | 6 | 8 | 2 | 4.7 | 6.3 | 1.7 | 3.0 | 2.3 | 0.7 |

BEF = before addition of water;
AFT = after addition of water;
Δ = olfactory intensity (BEF − AFT)
Scale of olfactory intensity: 0 to 10 (0 = odorless; 10 = very intense/saturated odor)

It was thus observed at T0 that the aerosol of Example 1 comprising the perfume capsules according to the invention has no odor before the addition of water, in contrast with the aerosols C1 and C2 (outside the invention), which shows that the perfume capsules in aerosols C1 and C2 are not leaktight even before the addition of water.

It was also observed, both at 4 hours and at 24 hours, that the aerosol of Example 1 after stimulation with water, gave a more intense odor than that observed for aerosols C1 and C2, which shows greater release of perfume in response to the water stimulus.

### EXAMPLE 2: ANTIPERSPIRANT AND DEODORANT PRODUCT

An anhydrous antiperspirant aerosol having the following composition was prepared:

| Ingredients | (weight %) |
|---|---|
| Dimethicone | 23.26 |
| Isopropyl palmitate | 4.80 |
| Dimethicone dimethiconol | 8.80 |
| Triethyl citrate | 5.60 |
| Modified bentonite | 2.08 |
| Anhydrous aluminum | 28.00 |
| hydroxychloride | |
| Perfume capsules of | 26.40 |
| Example A | |
| Free perfume | 1.06 |
| Total | 100.00 |

The perfume capsules of Example A may be replaced with the capsules of Examples B to H described previously.

46.5 g of dimethicone, 9.6 g of isopropyl palmitate, 17.6 g of dimethicone/dimethiconol, 11.2 g of triethyl citrate and 2.13 g of perfume are placed in a tank equipped with a stirrer and a bottom turbomixer. The mixture is homogenized with vigorous stirring (1000 rpm). 4.16 g of modified bentonite are then added and the mixture is left to stand for 10 minutes. The mixture is homogenized again with vigorous stirring (1200 rpm), followed by addition of 56.0 g of anhydrous aluminum hydroxychloride. The mixture is homogenized again with vigorous stirring (1200 rpm), followed by addition of 52.8 g of the capsules of Example A. A thick opaque beige-colored liquid was thus obtained, which is introduced into a closed aerosol can. The air is removed from the container, which is then filled with the propellant gas (isobutane) in a 15 (fluid)/85 (gas) ratio.

About 1.0 g of composition of Example 2 was applied onto the skin. After 1 minute, the perfume odor intensity was evaluated (APPLICATION) and graded from 0 to 10. 2, 4 and 6 hours later, the perfume odor intensity was re-evaluated again (BEF) before adding about 0.1 g of water (three sprays) to the composition applied to the skin. After waiting for 30 seconds, the perfume odor intensity was re-evaluated (AFT).

| | | | | | | | | Odor | | |
|---|---|---|---|---|---|---|---|---|---|---|
| | | Odor | | | Odor | | | intensity | | |
| | Odor intensity T0 | intensity T2 h | | | intensity T4 h | | | T6 h | | |
| Product | APPLICATION | BEF | AFT | Δ | BEF | AFT | Δ | BEF | AFT | Δ |
| Example | 7.0 | 3.75 | 7.5 | 3.75 | 3.5 | 6.5 | 3.0 | 2.5 | 4.5 | 2.0 |
| 2 | | | | | | | | | | |

BEF = before addition of water;
AFT = after addition of water
Δ = amplitude of difference in intensity of perfume odor (BEF − AFT)
Scale of perfume odor intensity: 0 to 10 (0 = odorless; 10 = very intense/saturated odor).

It was thus observed at T0 that the composition of Example 2 has a strong odor which decreases rapidly at T2h, T4h and T6h after application. It was also observed that the spraying of water onto the product at T2h, T4h and T6h leads to an increase in the odor intensity (especially of the fresh notes), which demonstrates substantial release of perfume each time.

### EXAMPLE 3: DRY SHAMPOO PRODUCT

An anhydrous dry shampoo having the following composition was prepared:

| Ingredients | (weight %) |
|---|---|
| Calcium carbonate | 14.8 |
| Corn starch Dry-Flo | 75.0 |
| (Akzo Nobel) | |
| Distearylammonium- | 2.0 |
| modified hectorite | |
| Isopropyl myristate | 3.2 |
| Perfume capsules of | 5.0 |
| Example F | |
| Total | 100.00 |

The perfume capsules of Example F may be replaced with the capsules of Examples A to E, G and H described previously.

14.8 g of calcium carbonate, 75.0 g of corn starch, 2.0 g of modified hectorite and 3.2 g of isopropyl myristate are placed in a tank equipped with a stirrer and a bottom turbomixer. The mixture is turbomixed and stirred for three minutes at 1500 rpm. 5.0 g of capsules of Example F are then added while homogenizing the mixture. The mixture thus obtained is introduced into an aerosol device. The air is removed from the container, which is then filled with the propellant gas (isobutane) in a 15 (fluid)/85 (gas) ratio.

After spraying the composition onto the hair, it is noted, when the person perspires or on contact with sebum, that perfume is released in the course of the day.

### EXAMPLE 4: DRY SHAMPOO PRODUCT

An anhydrous dry shampoo having the following composition was prepared:

| Ingredients | (weight %) |
|---|---|
| Calcium carbonate | 14.8 |
| Corn starch Dry-Flo | 76.4 |
| (Akzo Nobel) | |
| Distearylammonium- | 2.0 |
| modified hectorite | |
| Isopropyl myristate | 3.0 |
| Perfume capsules of | 5.0 |
| Example B | |
| Free perfume | 0.8 |
| Total | 100.00 |

The perfume capsules of Example B may be replaced with the capsules of Examples A and C to H described previously.

14.8 g of calcium carbonate, 76.4 g of corn starch, 2.0 g of modified hectorite, 3.0 g of isopropyl myristate and 0.8 g of free perfume are placed in a tank equipped with a stirrer and a bottom turbomixer. The mixture is turbomixed and stirred for three minutes at 1500 rpm with the paddles and turbomixer. 3.0 g of capsules of Example B are then added while homogenizing the mixture with the paddles. The mixture obtained is introduced into an aerosol device. The air is removed from the container, which is then filled with the propellant gas (isobutane) in a 15 (fluid)/85 (gas) ratio.

After spraying the composition onto the hair, it is noted, when the person perspires or on contact with sebum, that perfume is released in the course of the day.

### EXAMPLE 5: ANTIPERSPIRANT AND DEODORANT PRODUCT

An anhydrous antiperspirant aerosol having the following composition was prepared:

| Ingredients | (weight %) |
|---|---|
| Dimethicone | 23.26 |
| Isopropyl palmitate | 4.80 |
| Dimethicone dimethiconol | 8.80 |
| Triethyl citrate | 5.60 |
| Modified bentonite | 2.08 |
| Anhydrous aluminum | 28.00 |
| hydroxychloride | |
| Perfume capsules of | 26.40 |
| Example H | |
| Free perfume | 1.06 |
| Total | 100.00 |

The perfume capsules of Example H may be replaced with the capsules of Examples A to G described previously.

46.5 g of dimethicone, 9.6 g of isopropyl palmitate, 17.6 g of dimethicone/dimethiconol, 11.2 g of triethyl citrate and 2.13 g of perfume are placed in a tank equipped with a stirrer and a bottom turbomixer. The mixture is homogenized with vigorous stirring (1000 rpm). 4.16 g of modified bentonite are then added and the mixture is left to stand for 10 minutes. The mixture is homogenized again with vigorous stirring (1200 rpm), followed by addition of 56.0 g of anhydrous aluminum hydroxychloride. The mixture is homogenized again with vigorous stirring (1200 rpm), followed by addition of 52.8 g of the capsules of Example H. A thick opaque beige-colored liquid was thus obtained, which is then introduced into a closed aerosol can. The air is removed from the container, which is then filled with the propellant gas (isobutane) in a 15 (fluid)/85 (gas) ratio.

After spraying the composition onto the armpits, it is noted that, on contact with sweat, the perfume of the deposited composition is released and the olfactory effect lasts throughout the day.

1. An anhydrous composition comprising:
1) at least particles comprising a core containing at least one beneficial agent and an envelope surrounding the core; said envelope comprising at least one hydrophobically modified polysaccharide and at least one water-soluble carbohydrate and/or water-soluble polyol;
said particles simultaneously having a poured powder density ranging from 300.0 g/l to 600.0 g/l and an absolute density of greater than 1.0 and
2) at least one propellant.

2. The composition as claimed in claim 1, comprising a physiologically acceptable medium.

3. The composition as claimed in claim 1, in which the particles are spherical and, in particular have a number-mean diameter ranging from 1 to 30 μm and a volume-mean diameter ranging from 5 to 150 μm.

4. The composition as claimed in claim 1, in which the hydrophobically modified polysaccharide is chosen from celluloses and hydrophobically modified derivatives thereof, starches and hydrophobically modified derivatives thereof, guars and hydrophobically modified hydroxyethyl-, carboxymethyl- and hydroxypropyl-guar derivatives; hydrophobically modified dextrans; hydrophobically modified pullulans, hydrophobically modified inulins.

5. The composition as claimed in claim 1, in which the hydrophobically modified polysaccharide represents from 25% to 80% by relative to the total weight of the envelope of the particle.

6. The composition as claimed in claim 1, in which the water-soluble polyol is chosen from triols, tetraols, pentols, hexols, heptols, octaols and nonanols, and mixtures thereof.

7. The composition as claimed in claim 1, in which the water-soluble carbohydrate is chosen from water-soluble monosaccharides, water-soluble oligosaccharides and water-soluble polysaccharides.

8. The composition as claimed in claim 7, in which
the monosaccharides are chosen from tetroses, pentoses, hexoses and heptoses;
the oligosaccharides are chosen from diholosides, triholosides, glucose syrups and glucose-fructose syrups, and more particularly glucose syrups with a D.E. ranging from 21 to 60;
the polysaccharides are chosen from dextrans, pullulans and maltodextrins.

9. The composition as claimed in claim 7, in which the water-soluble carbohydrate is chosen from water-soluble oligo- and polysaccharides.

10. The composition as claimed in claim 1, in which the water-soluble carbohydrate(s) and/or the water-soluble polyols represent from 20% to 75% by weight relative to the total weight of the envelope of the particle.

11. The composition as claimed in claim 1, in which the envelope of the particles with release of beneficial agent is formed from at least one starch (C₅-C₂₀)alkenyl succinate and from at least one maltodextrin with a D.E. ranging from 4 to 20 and/or a glucose syrup with a D.E. ranging from 21 to 60.

12. The composition as claimed in claim 11, in which the envelope of the particles with release of beneficial agent is formed from
a) at least one starch (C₅-C₂₀)alkenyl succinate in an amount ranging from 25% to 80% by weight relative to the total weight of the envelope of the particle and
b) at least one maltodextrin with a D.E. ranging from 4 to 20 and/or a glucose syrup with a D.E. ranging from 21 to 60, in an amount ranging from 20% to 75% by weight relative to the total weight of the envelope of the particle.

13. The composition as claimed in claim 1, in which the particles with release of beneficial agent may be obtained according to a process comprising at least the following steps:
an aqueous solution formed from a mixture of the water-soluble carbohydrate and/or the water-soluble polyol and of the hydrophobically modified polysaccharide is prepared, the beneficial agent is then added and the whole is stirred so as to form an emulsion; and
said emulsion thus formed is homogenized at high pressure at a pressure ranging from 10 to 200 bar;
said emulsion is sprayed in a drying chamber; and
the water is extracted for a time not exceeding 3 hours, with a fluid under pressure so as to obtain particles with release of beneficial agent.

14. The composition as claimed in claim 1, in which the beneficial agents are chosen from:
(i) fatty substances;
(ii) fragrancing substances;
(iii) pharmaceutical active principles;
(iv) cosmetic active agents.

15. The composition as claimed in claim 1, in which the beneficial agents are chosen from fragrancing substances.

16. The composition as claimed in claim 1, in which the particles comprise at least one or more fragrancing substances with a saturating vapor pressure at 25° C. of greater than or equal to 10.0 Pa and said fragrancing substance(s) represent from 50% to 100% by weight relative to the total weight of the fragrancing substances present in the particles.

17. The composition as claimed in claim 1, wherein
a) the particles comprise at least one fragrancing substance and
b) the composition also comprises at least one fragrancing substance in free form, which may be identical to or different from said fragrancing substance present in said particles.

18. The composition as claimed in claim 1, wherein it exclusively contains one or more fragrancing substances encapsulated in the particles.

19. The composition as claimed in claim 1, comprising at least one deodorant active agent and/or at least one antiperspirant active agent in free form and/or in encapsulated form.

20. An aerosol device formed from a container comprising a composition as defined and as claimed in claim 1 and from a means for dispensing said composition.

21. A cosmetic process for caring for and/or for the hygiene of and/or for conditioning and/or for fragrancing and/or for making up a keratin material, which comprises applying to said keratin material a composition as claimed in claim 1.

22. A cosmetic process for treating body odor and optionally human perspiration, which applying to a keratin material a composition as claimed in claim 19.

23. A consumer product, which is conditioned in an aerosol device formed from a container comprising a composition as claimed in claim 1 and from a means for dispensing said composition.
